(19) United States
(12) Patent Application Publication
(10) Pub. No.: US 2014/0335083 A1
(43) Pub. Date: Nov. 13, 2014

(54) METHODS OF TREATMENT AND PREVENTION OF EYE DISEASES

(71) Applicant: GLAXO GROUP LIMITED, Brentford, Middlesex (GB)

(72) Inventors: Peter Adamson, Stevenage (GB); David Shima, London (GB); Yin Shan Eric Ng, London (GB)

(73) Assignee: Glaxo Group Limited, Brentford, Middlesex (GB)

(21) Appl. No.: 14/361,397

(22) PCT Filed: Nov. 30, 2012
(86) PCT No.: PCT/EP2012/074156

§ 371 Date: May 29, 2014

Related U.S. Application Data
(60) Provisional application No. 61/565,707, filed on Dec. 1, 2011.

(51) Int. Cl. C07D 295/145; A61K 31/5375; A61K 45/06

(52) U.S. Cl. CPC C07D 295/145; A61K 45/06; A61K 31/5375
USPC 424/133.1; 514/237.8

(57) ABSTRACT

The present invention provides compositions and methods useful for treating and preventing neovascular AMD by inhibition of CCR3. The compositions and methods are useful for treating and preventing diseases and disorders such as but not limited to, neovascular AMD

### FIELD OF THE INVENTION

The present invention relates generally to methods for the treatment and/or prevention of eye diseases, and more particularly to treatment and/or prevention of eye diseases, such as neovascular age-related macular degeneration, secondary to dry (atrophic) macular degeneration, using agents that inhibit the expression and/or activity of CCR3 or VEGF and VEGF signaling inhibitors, alone or in combination.

### BACKGROUND OF THE INVENTION

Neovascularization, also called angiogenesis, is the process of forming new blood vessels. Neovascularization occurs during normal development, and also plays an important role in wound healing following injury to a tissue. However, neovascularization has also been implicated as an important cause of a number of pathological states including, for example, cancer, rheumatoid arthritis, atherosclerosis, psoriasis, and diseases of the eye including diabetic retinopathy, diabetic macular edema, and neovascular AMD. Eye diseases associated with vascular leaking and/or neovascularization are responsible for the vast majority of visual morbidity and blindness in developed countries (Campochiaro (2004) Expert Opin. Bioi. Ther.4:1395-402). Eye disorders associated with ocular neovascularization and increased vascular permeability are a major cause of vision loss and blindness.

Age-related macular degeneration (AMD) is the leading cause of blindness in the developed world. There are two major clinical presentations of AMD, atrophic (dry AMD) and wet AMD. Atrophic AMD is characterised by the degeneration of retinal pigment epithelial (RPE) and neuroretina. The early stages of atrophic AMD are associated with the formation of drusen, under the RPE cell layer. Early atrophic AMD can progress to an end stage disease where the RPE degenerates completely and forms sharply demarcated areas of RPE atrophy in the region of the macula: “geographic atrophy”. In this form of the disease, the degeneration of RPE results in the secondary death of macular photoreceptors and in these cases leads to the severe vision loss.

Approximately 10-20% of AMD patients suffering from dry or geographic atrophy AMD develop subsequent choroidal neovascularization, (CNV). This form of the disease is known as “wet AMD” and can be associated with some of the most severe vision loss. In wet AMD, new choroidal vessels (neovessels) proliferate in the choroid or can punch through Bruch's membrane and proliferate into and under the RPE and neuroretina (see, for example, Campochiaro et al. (1999) Mol. Vis. 5:34). In typical cases, atrophic AMD develops in the eye before the development of the wet form, however, on infrequent occasions, the neovascular or wet form can develop in the absence of prior development of the atrophic form. In both forms of the disease, vision loss occurs due to the death of photoreceptor cells, although in wet AMD fluid leak and occasional internal bleeding from the leaky vessels (increased vascular permeability) formed during CNV also causes vision loss.

For AMD there has been progress in developing novel treatments to address some aspects of wet AMD, in particular the reduction of leaky vessel bleeding from CNV by various molecules that inhibit either vascular endothelial growth factor (VEGF) or the VEGF receptor signalling pathway.

Chemokines are a large family of small proteins which are involved in trafficking and recruitment of leukocytes (for review see, Luster, New Eng. J. Med., 338, 436-445 (1998)). They are released by a wide variety of cells and act to attract and activate various cell types, including eosinophils, basophils, neutrophils, macrophages, T and B lymphocytes. There are two major families of chemokines, CXC-(α) and CC-(β) chemokines, classified according to the spacing of two conserved cysteine residues near to the amino terminus of the chemokine proteins. Chemokines bind to specific cell surface receptors belonging to the family of G-protein-coupled seven transmembrane-domain proteins (for review see Luster, 1998). Activation of chemokine receptors results in, amongst other responses, an increase in intracellular calcium, changes in cell shape, increased expression of cellular adhesion molecules, degranulation and promotion of cell migration (chemotaxis).

To date, 9 members of CC chemokine receptors have been identified (CCR-I to 9). Of particular importance to the current invention is the CC-chemokine receptor-3 (CCR-3), which is predominantly expressed on eosinophils, and also on basophils, mast cells and Th2 cells (Luster, 1998). Chemokines that act at CCR-3, such as RANTES, MCP-3 and MCP-4, are known to recruit and activate eosinophils.

It had previously been shown that human microvascular endothelial cells express CCR3 (Salcedo et al., 2001) and that agonists active at the CCR3 receptor could function as chemotactic agents for vascular endothelia. This process could lead to promotion of vascular angiogenesis through this chemotactic effect on vascular endothelial cells in both chick chorioallantoic membranes and aortic rings (Salcedo et al., 2001).

These observations were subsequently extended to studies in AMD (Takeda et al., 2009) which demonstrated that intravitreal injection of anti-CCR3 antibodies, anti-CCR3 agonist antibodies, or pharmacological inhibitors of CCR3 were effective in limiting the extent of CNV following laser induction in mice. These data were confirmed in lasered mice with homozygous gene deficiency in either CCR3 or specific CCR3 agonist genes. Other data also showed that the CCR3 agonists CCL11 and CCL24 were induced following laser-induction of CNV. (Takeda et al., 2009).

Extension of these studies into mice which are deficient in the production of eosinophils or mast cells, the cells most closely associated with the expression of CCR3, demonstrated that these mice retained the ability to generate CNV in response to laser photocoagulation and were no less sensitive to intravitreal CCR3 antagonism in blocking this disease. The studies strongly suggest that the mechanism of action of CCR3 in limiting the generation of laser-induced CNV is not through an action on systemic eosinophils or mast cells. The fact that intravitreal anti-CCR3 therapies were used to control disease in these models is also supportive of a more local effect in the eye. Studies also suggested that anti-CCR3 therapy does not limit the recruitment of macrophages or neutrophils into laser-induced CNV lesions (Takeda et al., 2009).

A number of additional independent studies have now confirmed these findings in mouse models of CNV AMD; specifically oral dosing of YM-344031 was effective in a laser-induced model of CNV AMD (Mizutani et al., 2011). However when CCR3 antagonists are dosed to the sub-retinal space, such agents appear incapable of restricting the angiogenesis induced by co-administration of matrigel, although anti-VEGF therapeutics were effective in this model (Li et al., 2010).

In other studies, CCR3 neutralisation using an anti-CCR3 antibody did not alter the amount of VEGF generated in the eye in response to laser, suggesting that the actions of CCR3 and VEGF in controlling CNV AMD could be independent. Similarly intravitreal injection of a neutralising anti-VEGF mAb had no effect on the expression of CCR3 present on choroidal endothelial cells further supporting this hypothesis of independent actions in controlling CNV in rodents (Takeda et al., 2009).

Studies examining corneal pathologic angiogenesis also observed that injury/prostaglandin E2 induced angiogenesis was independent of VEGF but correlated with eotaxin, again suggesting that the eotaxin/CCR3 axis could be an independent mechanism to VEGF in controlling angiogenesis (Liclican et al., 2010).

It is generally accepted that anti-VEGF therapeutics control CNV in both man and animal models by reducing the vascular permeability of newly formed choroidal vessels which have entered the retina rather than a direct action on angiogenesis. Studies have also suggested that in cultures of human coronary artery endothelial cells permeability of in vitro cultures could be increased by agonists of CCR3 (eotaxin) (Jamaluddin et al., 2009) presenting the possibility that CCR3 antagonism generally impacts the vascular permeability of ocular vessels.

Initial studies by Takeda et al., 2009 showed that CCR3 was expressed in human CNV retina but not in retina which had only atrophic AMD, without clinical evidence of progression to CNV disease. The expression was co-localised with CD31 (PECAM-1) a marker of choroidal vasculature and the specificity to blood vessels in CNV AMD was confirmed by the absence of staining in both retinal fibrosis and melanoma (Takeda et al., 2009). The natural CCR3 agonist eotaxin-1 (CCL11), eotaxin-2 (CCL24) and eotaxin-3 (CCL26) were also all found to be expressed in the stroma and co-localised with blood vessels in surgically excised human choroidal neovascular AMD tissue (Takeda et al., 2009). In models similar to mouse models there is no evidence to date that eosinophils or mast cells are associated with human AMD and in fact attempts to locate such cells in human CNV AMD lesions has not yielded any significant findings (Takeda et al., 2009). Additionally eotaxin was identified as a potential serum biomarker for human AMD (Mo et al. 2010) in which it was found to be significantly associated (p<−0.02-p<0.005) with type I and II AREDS patients and atrophic AMD but interestingly not with CNV AMD, although examination of post-mortem human eyes in this same study suggested a correlation with early AMD, atrophic AMD and CNV AMD with the strongest staining associated with the neovascular endothelium in CNV AMD. Eotaxin was also found to be significantly associated with vascularly active disease versus vascularly inactive disease in a comparison of vitreal samples from retinopathy of prematurity patients suggesting a potential CCR3 involvement in promoting angiogenesis in the retinal circulation (Sato et al., 2009).

Therefore, it has previously been demonstrated that AMD develops mechanistically from two different and unrelated pathways, angiogenesis and vascular permeability. It has been shown that AMD can be treated with an anti-VEGF inhibitor (WO 2007/064752). It has also been shown that intravitreal treatment with a CCR3 antagonist can affect the size of a CNV lesion (Takeda et al., 2009).

Surprisingly, applicants have discovered that CCR3 antagonism has an effect on specifically reducing vascular permeability and angiogenesis on choroidal neovessels as distinct from neo retinal vessels induced by ectopic retinal VEGF production or induced by hyperbaric oxygen treatment, and therefore is useful for specifically treating/preventing, (including slowing the progression of the disease and/or its symptoms), choroidal vascular permeability associated with neovascular AMD.

Applicants have also discovered that blocking both the angiogenesis pathway and the permeability pathway of the disease, produces a functional response in vivo. Therefore, treatment with a combination of an anti-VEGF inhibitor and a CCR3 inhibitor is an effective combinatorial treatment and/or preventative for neovascular AMD, where both agents are able to independently effect lesion growth and vascular permeability and produce an additive effect.

There remains a need for new methods of treating ocular neovascular disorders, in particular neovascular AMD. The present invention is directed to such a need.

### SUMMARY OF THE INVENTION

The present invention relates to methods for treatment and/or prevention of eye diseases and disorders by inhibition of CCR3, for example inhibition of expression and/or activity of CCR3 protein. In particular embodiments, eye diseases amenable to treatment and/or prevention by the methods of the present invention are associated with neovascularization and increased vascular permeability. Specifically, such diseases include, for example, but are not limited to, age-related macular degeneration, and the like.

In one embodiment, the methods as disclosed herein comprise administering to a subject in need of treatment and/or prevention of an eye disease, a pharmaceutical composition comprising an agent which inhibits CCR3, for example an agent which inhibits the expression of CCR3 and/or the activity of CCR3 protein. It is not intended that the present invention to be limited to any particular stage of the disease (e.g., early or advanced).

In some embodiments, the present invention provides methods to inhibit CCR3 by blocking associated enzyme activity and all downstream effectors of CCR3 activation.

In some embodiments, prevention and/or reduction of choroidal neovascular permeability leads to prevention and/or reduction of symptoms (i.e., prevents progression of the disease) associated with neovascular AMD.

In another embodiment, the present invention provides methods of treating and/or preventing AMD in a subject with, or at risk of AMD, comprising administering to the subject a pharmaceutical composition comprising an agent which inhibits the activity and/or expression of CCR3 protein, wherein inhibition of the CCR3 protein reduces the progression or stops a symptom of AMD.

In another embodiment, the present invention provides methods of treating and/or preventing a subject with, or at risk of AMD, comprising administering to the subject a pharmaceutical composition comprising an agent which inhibits the activity and/or expression of CCR3 protein, wherein inhibition of the CCR3 protein reduces the progression or stops a symptom of AMD, and further administering in combination with the agent, an anti-VEGF inhibitor or a VEGF signaling inhibitor.

In another embodiment, the present invention provides methods of preventing the development of a CNV lesion on an atrophic retinal background in a subject with dry or geographic atrophy AMD, comprising administering to the subject a pharmaceutical composition comprising an agent which inhibits the activity and/or expression of CCR3 protein, wherein inhibition of the CCR3 protein prevents the development of such lesion, which agent can be administered alone or in combination with an anti-VEGF inhibitor.

In another embodiment, the present invention provides methods of preventing the transition/progression of atrophic and non-vascular AMD to neovascular AMD comprising administering to the subject a pharmaceutical composition comprising an agent which inhibits the activity and/or expression of CCR3 protein, wherein inhibition of the CCR3 protein such transition to neovascular AMD, which agent can be administered alone or in combination with an anti-VEGF inhibitor.

In some embodiments, the agent that inhibits CCR3 can inhibit the expression of CCR3, for example inhibit the translation of CCR3 RNA to produce the CCR3 protein. In alternative embodiments, the agent that inhibits CCR3 can inhibit CCR3 protein activity. Any agent is encompassed for use in the methods as disclosed herein. In some embodiments, the agent can be a small molecule, nucleic acid, nucleic acid analogue, protein, antibody, peptide, aptamer or variants or fragments thereof. In some embodiments, the agent is a nucleic acid agent, for example, an RNAi agent, for example, an siRNA, shRNA, miRNA, dsRNA or ribozyme or variants thereof.

In some embodiments, the agent that inhibits the protein activity of CCR3 is a small molecule, for example, but not limited to, a small molecule reversible or irreversible inhibitor of CCR3 protein. In some embodiments, such a small molecule is a morpholin-acetamide-based compound. In some embodiments, a small molecule inhibitor of CCR3 is, for example, but not limited to, 4-[[[[[[(2s)-4-[(3,4-dichlorophenyl)methyl]-2-morpholinyl]methyl]-amino]carbonyl]-amino]methyl]benzamide, or a pharmaceutically acceptable salt thereof (CCR3 inhibitor '994). See U.S. Pat. Nos. 7,157,457 and 7,531,651.

In another embodiment, such a small molecule is a morpholine urea-based compound. In some embodiments, a small molecule inhibitor of CCR3 is, for example, but not limited to, N-[[(2S)-4-[(3,4-difluorophenyl)methyl]-2-morpholinyl]methyl]-3-[(methylsulfonyl)amino]-benzeneacetamide, or a pharmaceutically acceptable salt thereof (CCR3 inhibitor '575). See U.S. Pat. No. 7,101,882.

In some embodiments where a subject is administered a pharmaceutical composition comprising an inhibitor of CCR3, the methods can further comprise administering to the subject additional therapeutic agents, for example but not limited to therapeutic agents used in the treatment of eye diseases, including AMD, and the like. It will be understood that the administration of therapeutic agents for treating ocular diseases may involve the application of certain procedures, for example, but not limited to, retinal focal laser photocoagulation, pan-retinal photocoagulation, intravitreal administered steroids, such as triamcinolone, intravitreal steroid implants containing fluocinolone acetonide, and intravitreal administered anti-VEGF therapeutics such as pazopanib, Lucentis®, Avastin®, and Aflibercept®.

In some embodiments, the methods as disclosed herein for the treatment and/or prevention of neovascular eye diseases or disorders are applicable to subjects, for example mammalian subjects. In some embodiments, the subject administered an agent that inhibits the activity or expression of the CCR3 protein is a human.

### BRIEF DESCRIPTION OF FIGURES

FIG. 1 shows group quantitation of CNV in C57B16 mice by fluorescence angiography following systemic treatment with GW766994. Upper panel shows mean CNV lesion size per eye with corresponding 95% confidence limits. Lower panel shows examples of fundoscopy image assessed by direct fluorescence angiography of mouse retina following treatment of mice with the CCR3 antagonist 4-[[[[[[(2s)-4-[(3,4-dichlorophenyl)methyl]-2-morpholinyl]methyl]-amino]carbonyl]-amino]methyl]benzamide (CCR3 inhibitor '994) (2-30 mg/kg QD po) and the spectrum selective kinase inhibitor pazopanib (5-[[4-[(2,3-dimethyl-2h-indazol-6-yl)methylamino]-2-pyrimidinyl]amino]-2-methylbenzenesulfonamide) (20 mg/kg QD po) at 1 week and 2 weeks following laser-induction of CNV.

FIG. 2 shows group quantitation of mean CNV with corresponding 95% confidence limits in C57B16 mice by fluorescence angiography following treatment of mice with the CCR3 inhibitor '994 (8-30 mg/kg QD and 8 mg/kg BID po) and the spectrum selective kinase inhibitor pazopanib (20 mg/kg QD po) at 1 week and 2 weeks following laser-induction of CNV.

FIG. 3 shows group quantitation of mean CNV in JR5558 mice by fluorescence angiography following systemic treatment with CCR3 inhibitor '994. Upper panel shows total mean CNV lesion area per eye with corresponding 95% confidence limits. Middle panel shows total number of CNV lesions per eye with corresponding 95% confidence limits, and the lower panel show direct fluorescence angiograms of mouse retina following treatment of mice with the CCR3 inhibitor '994 (2-30 mg/kg QD). Compound was dosed for 12 days between P14 and P26.

FIG. 4 shows group quantitation of CNV in JR5558 mice by fluorescence angiography following systemic treatment with 8 mg/kg CCR3 inhibitor '994 BID. Upper panel shows total mean CNV lesion area per eye with corresponding 95% confidence limits. Middle panel shows total number of CNV lesions per eye with corresponding 95% confidence limits, and the lower panel show direct fluorescence angiograms of mouse retina following treatment of mice with the CCR3 inhibitor '994 (8 mg/kg BID). Compound was dosed for 12 days between P14 and P26.

FIG. 5 shows histochemical detection of vascular CNV in the choroid (retina removed) of JR5558 with isolectin B4 staining following treatment of mice with the CCR3 inhibitor '994 (8-30 mg/kg QD). Compound was dosed for 12 days between P14 and P26.

FIG. 6 shows histochemical detection of vascular CNV in the choroid (retina removed) of JR5558 with isolectin B4 staining following treatment of mice with the CCR3 inhibitor '994 (8-30 mg/kg QD). Compound was dosed for 12 days between P14 and P26.

FIG. 7 shows group quantitation of CNV in JR5558 mice by fluorescence angiography topical treatment with 5 ul 1-10 mg/ml CCR3 inhibitor '994 BID dosed to both eyes and comparison with 8 mg/kg CCR3 inhibitor '994 i.p. BID dosed systemically. Upper panel shows total mean CNV lesion area per eye with corresponding 95% confidence limits. Lower panel shows total number of CNV lesions per retina with corresponding 95% confidence limits, following treatment of mice with the CCR3 inhibitor '994 applied either topically or systemically (8 mg/kg i.p. BID). Compound was dosed for 12 days between P14 and P26.

FIG. 8 shows group quantitation of CNV in JR5558 mice by fluorescence angiography following systemic dosing with either vehicle, 100 ug anti-VEGFR2 i.p. QD, 30 mg/kg CCR3 inhibitor '994 i.p. QD, 100 ug anti-VEGFR2 i.p. QD plus 30 mg/kg CCR3 inhibitor '994 i.p QD and 50 ug anti-VEGFR2 i.p. QD plus 30 mg/kg CCR3 inhibitor '994 i.p. QD. Upper panel shows total CNV lesion area per retina with corresponding 95% confidence limits. Lower panel shows total number of CNV lesions per retina with corresponding 95% confidence limits CCR3 inhibitor '994 was dosed for 12 days between P14 and P26. Anti-VEGFR2 was dosed for 6 days from P14 and a further 5 days from P19.

FIG. 9 shows group quantitation of vascular permeability of individual CNV lesions in JR5558 mice by fluorescence angiography following systemic dosing with either vehicle, 100 ug anti-VEGFR2 i.p. QD, 30 mg/kg CCR3 inhibitor '994 i.p. QD, or 100 ug anti-VEGFR2 i.p. QD plus 30 mg/kg GW766994 i.p QD. CCR3 inhibitor '994 and anti-VEGR2 as well as combinations were dosed for 2 days between P24 and P26.

FIG. 10 shows analysis of grade IV lesions in right and left eyes of individual Cynomolgus monkeys following CNV induction by laser photocoagulation and dosed 1 day prior to laser with vehicle, 20 mg/kg GW782415X ((S)-1-((4-(3,4-dichlorobenzyl) morpholin-2-yl)methyl)-3-((2-methyl-2H-tetrazol-5-yl)methyl)urea) (hereinafter “the '415 compound”) po TID or 3 mg/kg TID po for 16, 24 or 30 days, subsequently.

FIG. 11 shows the concentration response curve of eotaxin-1 on eosinophil shape change in Cynomolgus whole blood and the effect of pre-incubation of 10 nM and 100 nM the '415 compound is shown in appendix 1. Schild analysis determined a mean pA2 value of 7.8 for the '415 compound on eotaxin-1 stimulated eosinophil shape change in Cynomolgus whole blood.

FIG. 12 shows the effect of group quantitation of retinal angiogenesis (neovascularization) and the effect of 8 mg/kg i.p. BID following exposure of C57B16 mice to hyperbaric oxygen (oxygen induced retinopathy model).

FIG. 13 shows the effect of group quantitation of retinal angiogenesis (neovascularization) and the effect of 10 mg/ml dosed topically and SU4312 (5 ug every 5 days, peri-ocularly) to one eye BID following exposure of C57B16 mice to hyperbaric oxygen (oxygen induced retinopathy model).

### DETAILED DESCRIPTION OF THE INVENTION

The inventors have discovered that CCR3 inhibitors can be used in the treatment and/or prevention, including progression, of ocular diseases, in particular vascular permeability associated with neovascular AMD.

The inventors have discovered that CCR3 inhibitors can be used in combination with anti-VEGF therapeutics and VEGF signaling inhibitors for the treatment and/or prevention, including progression, of ocular diseases, in particular vascular permeability associated with AMD.

The inventors have also discovered that CCR3 inhibitors in combination with anti-VEGF inhibitors can specifically be used in the treatment and/or prevention, including progression, of ocular diseases, in particular choroidal neovascularization associated with AMD. These effects appear specific to the choroid since similar effects of CCR3 inhibitors cannot be demonstrated on retinal vessels undergoing neovascuarization following exposure to hyperbaric oxygen (oxygen-induced retinopathy).

The inventors have discovered that CCR3 inhibitors can be used in the treatment and/or prevention, including progression, of ocular diseases, in particular choroidal neovascuarisation in an atrophic retinal background e.g., patients suffering from dry or geographic trophy associated AMD.

The inventors have discovered that CCR3 inhibitors can be used in conjunction with anti-VEGF therapeutics and VEGF signaling inhibitors in the treatment and/or prevention, including progression, of ocular diseases, in particular choroidal neovascularization in an atrophic retinal background e.g., patients suffering from dry or geographic trophy associated AMD.

Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “disease” or “disorder” is used interchangeably herein, and refers to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, disorder, sickness, illness, complaint or affectation.

The terms “choroidal vascular permeability” or “vascular permeable are commonly referred to by persons in the art as “leaky vessels”. The terms are used interchangeably herein to refer to impaired choroidal vasculature and increased vascular permeability.

The term “agent” refers to any entity which is normally not present or not present at the levels being administered in the cell. Agent can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein inhibitor of CCR3 within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The term “inhibiting” as used herein means that the expression or activity of CCR3 protein or variants or homologues thereof is reduced to an extent, and/or for a time, sufficient to produce the desired effect, for example, wherein inhibition of the CCR3 protein reduces or stops a symptom of vascular permeability and/or choroidal neovascularization, etc. The reduction in activity can be due to affecting one or more characteristics of CCR3 including decreasing its catalytic activity or by inhibiting a co-factor of CCR3 or by binding to CCR3 with a degree of activity that is such that the outcome is that of treating or preventing an ocular disorder. In particular, inhibition of CCR3 can be determined using an assay for CCR3 inhibition, for example, but are not limited to by using the bioassay for CCR3 protein as disclosed herein.

The terms “patient”, “subject” and “individual” are used interchangeably herein, and refer to an animal, particularly a human, to whom treatment including prophylaxic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

As used herein, the term “treating” includes reducing, alleviating or preventing, including preventing the progression of, at least one adverse effect or symptom of a condition, disease or disorder associated with AMD. Preventing the progression of at least one adverse effect or symptom of a condition, disease or disorder associated with AMD, includes, but is not limited to, preventing the development of a CNV lesion on an atrophic retinal background in a subject at risk of developing choroidal neovascuarization and/or subsequent increased choroidal vascular permeability; and/or preventing the transition of atrophic and non-vascular AMD to neovascular AMD. Methods for measuring positive outcomes of treatment include, but are not limited to reduction or maintenance of sub-retinal edema, measured by optical coherence tomography, reduction in the loss or maintenance of vision, or the gain of vision as assessed by best corrected visual acuity. Enhanced vascular permeability and choroidal neovascuarisation are also determined by fundus fluorescence angiography.

The term “effective amount” as used herein refers to the amount of therapeutic agent of pharmaceutical composition to reduce, stop or prevent at least one symptom of the disease or disorder, for example a symptom or disorder of AMD. For example, an effective amount using the methods as disclosed herein would be considered as the amount sufficient to reduce or prevent a symptom of the disease or disorder, for example a complete or partial resolution and/or maintenance of AMD as measured by OCT or an increase and/or maintenance in best corrected visual acuity greater than 5 letters (as assessed by EDTRS eye chart), or a reduction in the size of the neovascuarisation or neovascular permeability as assessed by fundus fluorescence angiography. An effective amount as used herein would also include an amount sufficient to prevent or delay the development of macula edema, enhanced permeability, size of CNV lesion and associated vision loss. An effective amount as used herein would also include an amount sufficient to prevent or delay the development of a symptom of the disease, alter the course of a symptom disease (for example but not limited to, slow the progression of a symptom of the disease), or reverse a symptom of the disease.

As used herein, the terms preventing or prevention the development of a CNV lesion in a “subject at risk” of developing choroidal neovascularization and/or subsequent increased choroidal vascular permeability refers to e.g., a patient suffering from dry or geographic atrophy AMD.

As used herein, the terms “administering,” and “introducing” are used interchangeably and refer to the placement of the agents that inhibit CCR3 as disclosed herein into a subject by a method or route which results in at least partial localization of the agents at a desired site. The compounds of the present invention can be administered by any appropriate route which results in an effective treatment in the subject.

The articles “a” and “an” are used herein to refer to one or to more than one (i.e., to at least one) of the grammatical object of the article. By way of example, “an element” means one element or more than one element.

Agents that inhibit CCR3

In some embodiments, the present invention relates to the inhibition of CCR3. In some embodiments, inhibition is inhibition of nucleic acid transcripts encoding CCR3, for example inhibition of messenger RNA (mRNA). In alternative embodiments, inhibition of CCR3 is inhibition of the expression and/or inhibition of activity of the gene product of CCR3, for example the polypeptide or protein of CCR3, or isoforms thereof. As used herein, the term “gene product” refers to RNA transcribed from a gene, or a polypeptide encoded by a gene or translated from RNA.

In some embodiments, inhibition of CCR3 is by an agent. One can use any agent, for example but are not limited to nucleic acids, nucleic acid analogues, peptides, phage, phagemids, polypeptides, peptidomimetics, ribosomes, aptamers, antibodies, small or large organic or inorganic molecules, or any combination thereof. In some embodiments, agents useful in methods of the present invention include agents that function as inhibitors of CCR3 expression, for example inhibitors of mRNA encoding CCR3.

Other agents useful in the methods as disclosed herein as inhibitors CCR3 can be a chemicals, small molecule, large molecule or entity or moiety, including without limitation synthetic and naturally-occurring non-proteinaceous entities. In certain embodiments the agent is a small molecule having the chemical moieties as disclosed herein.

### Small Molecules

In some embodiments, agents that inhibit CCR3 are small molecules. Irreversible or reversible inhibitors of CCR3 can be used in the methods of the present invention.

CCR3 inhibitors effective in humans are commonly known by persons of ordinary skill and include those undergoing evaluation, for example undergoing pre-clinical and clinical assessment including Phase II clinical trials. A number of applications have been filed and published by SmithKline Beecham and its successor GlaxoSmithKline. Irreversible inhibitors of CCR3 are disclosed in WO 2002/26723A1, WO03/082293, U.S. Pat. Nos. 7,101,882, 7,157,457 7,531,651 and 7,560,548 which are specifically incorporated in their entirety herein by reference and disclose inter alia various series of morpholin-acetamide and morpholine urea compounds which are inhibitors of CCR3.

VEGF therapeutics effective in humans are commonly known by persons of ordinary skill and include those undergoing evaluation, for example undergoing pre-clinical and clinical assessment including Phase II clinical trials, and/or on sale, including pazopanib, Lucentis®, Avastin®, and Aflibercept®.

The compound 4-[[[[[[(2s)-4-[(3,4-dichlorophenyl)methyl]-2-morpholinyl]methyl]-amino]carbonyl]-amino]methyl]benzamide (CCR3 inhibitor '994), or a pharmaceutically acceptable salt or solvate thereof, is a particularly effective CCR3 inhibitor and is specifically useful in this invention.

The compound N-[[(2S)-4-[(3,4-difluorophenyl)methyl]-2-morpholinyl]methyl]-3-[(methylsulfonyl)amino]-benzeneacetamide (CCR3 inhibitor '575) or a pharmaceutically acceptable salt or solvate thereof, is a particularly effective CCR3 inhibitor and is specifically useful in this invention.

Other CCR3 inhibitors useful in the methods as disclosed herein are described in published patents and applications, WO 2002/26723A1, WO03/082293, U.S. Pat. Nos. 7,101,882, 7,157,457 7,531,651 and 7,560,548, and can be found using the CCR3 inhibition assays described therein.

All of the applications set out in the above paragraphs are incorporated herein by reference. It is believed that any or all of the compounds disclosed in these documents are useful for prophylaxis or treatment of AMD. The models described herein as exemplified in the Examples can be used by one of ordinary skill in the art to determine which of the disclosed compounds or other inhibitors of CCR3, for example antibodies, or RNAi are effective for the treatment or prevention of ocular diseases or disorders as claimed herein.

### EXAMPLES

The examples presented herein relate to the methods and compositions for the prevention and/or treatment of ocular neovascular disorders, and the like by inhibition of CCR3. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

In some embodiments, agents inhibiting CCR3 can be assessed in animal models disclosed herein for effect in reducing laser-induced CNV.

In some embodiments, agents inhibiting CCR3 can be assessed in animal models, for example, laser-induced choroidal neovascular AMD studies in the Cynomolgus monkey, disclosed herein.

In some embodiments, agents inhibiting CCR3, alone or in combination with an anti-VEGF inhibitor, can be assessed in animal models, for example, limiting the development of CNV in the JR5558 spontaneous model of CNV AMD.

### Example 1

Methods:

Laser-induced CNV in C57/BL6 mice

Adult 12 weeks old female C57BL/6 mice were used to generate the laser-induced CNV model. Mice were anesthetized by intraperitoneal ketamine hydrochloride (25 mg/kg) and xylazine (10 mg/kg) and their pupils dilated by topical tropicamide 1%. Laser-photocoagulation was performed a diode laser (680 nm; 210 mW power, 100 ms duration, 100 m spot diameter) under direct vision using a hand-held cover slide as a contact lens and were localized to the 2, 10, and 6 o'clock positions of the posterior pole of the retina in both eyes. These laser settings consistently generate a subretinal gas bubble which strongly correlates with laser-induced rupture of Bruch's membrane and successful induction of CNV. Only lesions in which a bubble is formed were included in the analysis. Fluorescein angiography using a Kowa Genesis small animal fundus camera was conducted at 1 week and 2 weeks following induction of CNV. The size of CNV lesions were quantified by digital image analysis the mean area of hyperfluorescence per lesion during the early phase (90 seconds post fluorescein injection). The pupils were dilated by topical tropicamide 1% and fluorescein sodium 2% administered by intraperitoneal injection (0.2 ml). Eyes were excluded if there was significant cataract or keratopathy that could affect laser energy delivery or angiography.

JR5558 CNV and Analyses

JR5558 mice develop CNV (on an atrophic background) spontaneously with CNV lesions first emerging approximately 12 days after birth. JR5558 mice were intraperitoneally treated with various amounts of a rat anti-mouse VEGF receptor (VEGFR)-2 blocking antibody (MAB4431; R&D systems) or a purified rat non-immune isotype match control IgG2α antibody (R&D systems) starting from P14 for a total of 10 doses in 11 days and CNV development was analyzed by fluorescein angiography (FA) 24 hours after the last dose on P25. For Mice treated with CCR3 inhibitor '994 they were dosed intraperitoneally or by eye drop with various concentrations of the drug starting at P14 for a total of 12 days and CNV development was analyzed by FA 24 hours after the last dose on P26. To study the combination effects of anti-VEGFR2 antibody and CCR3 inhibitor '994, mice were treated intraperitoneally with each material alone or in combination starting from P14 for a total of 10 doses in 11 days and CNV development was analyzed by FA 24 hours after the last dose on P25. Experiments exploring the effects of VEGF-A and CCR3 antagonists on vascular permeability from established CNV lesions, JR5558 mice utilised dosing for only two days after the development of CNV lesions and were treated with various amounts of the VEGFR-2 antibody or CCR3 inhibitor '994, or in combinations on P24 and P25. Fluorescein Angiography (FA) was performed on P26.

Fluorescein Angiography (FA) and image analysis

The pupils of mice were dilated with 2.5% tropicamide (Bausch & Lomb, Rochester, N.Y.) and 0.2 ml of 2% fluorescein sodium (Bausch & Lomb) diluted in water was administered by intraperitoneal injection. A Kowa Genesis-Df fundus camera (Kowa, Tokyo, Japan) was used to obtain fluorescein angiograms at early (90 seconds after fluorescein injection) and late (7 min) phases of dye transit. At the early phase, the vasculature of CNV tissue is clearly defined by the intravascular fluorescein dye. At the late phase, extravascular fluorescein is evident as patches of hyperfluorescence. To quantify the CNV area, Image J program was used to determine the size of each of the hyperfluorescence CNV in each eye with FA images from the early phase. Permeability of each CNV was determined by subtracting the hyper-fluorescent area of early phase (90 second post fluorescein injection) from late phase (7 minutes post fluorescein injection) FA images by using Image J.

Retinal whole-mount staining for CNV lesions

At 24 hours after FA, eyes were enucleated and fixed with 4% paraformaldehyde (PFA) in PBS for 3 hours at 4° C. Eyecup with or without retina removed was dissected, blocked in buffer containing 0.3% Triton X-100 and 5% FBS in PBS (blocking buffer) for 1 hour at room temperature, and incubated overnight at 4° C. with 0.5% fluorescein-isothiocyanate (FITC)-isolectin B4 (Vector, Burlingame, Calif.) at 1:200 dilution in blocking buffer. After 5 washes, the specimen was mounted with medium containing DAPI and viewed with an epifluorescence microscope.

Laser-Induced Choroidal Neovascular AMD studies in the Cynomolgus Monkey

Cynomolgus monkeys were dosed with vehicle or the CCR3 antagonist tool compound the '415 compound as shown in Table A for 29 days with laser occurring 1 day after laser photocoagulation. Fluorescence angiography analysis was conducted on days 14, 21 and 28 following laser.

TABLE A
Doses and dosing regimen of the ′415 compound used in
the laser-induced Cynomolgus Monkey CNV study

| | No. of | | | |
|---|---|---|---|---|
| | Animals | Dose Level^{a} | | Dose Concentration^{b} |
| Group | (males) | (mg/kg/dose) | (mg/kg/day) | (mg/mL) |
| 1 (Control)^{c} | 6 | 0 | 0 | 0 |
| 2 (Low) | 6 | 3 | 9 | 0.6 |
| 3 (High) | 6 | 20 | 60 | 4.0 |

^{a}Animals will be dosed three times daily approximately 8 hours apart at a volume of 5 mL/kg/dose on Days 1 through 30 of the dosing phase.
^{b}Dose concentrations are expressed in terms of parent compound and will be corrected for salt content using a correction factor of 1.015 for GW782415X.
^{c}Group 1 will receive vehicle control article only.

The macula of each eye underwent laser treatment with a 532-nm diode green laser burns (OcuLight GL, IRIDEX Corp Inc, Mountain View, Calif.) using a slit lamp delivery system and a Kaufman-Wallow (Ocular Instruments Inc, Bellevue, Wash.) plano fundus contact lens. Animals were anesthetized and nine areas symmetrically placed in the macula of each eye. The laser parameters included a 75-micron spot size and 0.1-second duration. The power setting used was assessed by the ability to produce a blister and a small haemorrhage. Unless haemorrhage is observed with the first laser treatment, a second laser spot will be placed adjacent to the first following the same laser procedure (except the wattage would be adjusted). For areas not adjacent to the fovea, the initial power setting was 500 mW; if a second spot is placed, the power was set to 650 mW. For the area adjacent to the fovea, the power settings was 400 mW (initial treatment) and 550 mW (second treatment). At the discretion of the retinal surgeon, power settings were adjusted based on observations at the time of laser.

Animals were fasted before fluorescein angiography for at least 2 hours prior to being anesthetized and the eyes dilated with a mydriatic agent. Animals were intubated due to the possibility of emesis following the fluorescein injection. Animals were subsequently given an intravenous injection of fluorescein. Images were taken at the start and end of the fluorescein injection. Following the fluorescein injection, a rapid series (approximately from dye appearance through 35 seconds) of stereo photographs of the posterior pole were taken of the right eye followed by a stereo pair of the posterior pole of the left eye. Additional stereo pairs were taken of both eyes approximately 1 to 2 and 5 minutes after fluorescein injection. Between approximately 2 and 5 minutes after fluorescein injection, nonstereoscopic photographs were taken of two mid-peripheral fields (temporal and nasal) of each eye. Grading Evaluation of fluorescein angiography was done by the contributing scientist for image evaluation according to the grading system shown in Table B. Grade IV lesions are considered clinically significant because these most closely resemble the active forms of classical choroidal neovascularization seen in various human retinal disorders, including age-related macular degeneration. Comparison of the incidence of Grade IV lesions between groups was evaluated.

TABLE B
Lesion Grading in Laser-Induced CNV in Cynomolgus Monkey

| Lesion Grade | Definition |
|---|---|
| I | No hyperfluorescence |
| II | Hyperfluorescence without leakage |
| III | Hyperfluorescence early or mid-transit and late leakage |
| IV | Bright hyperfluorescence early or mid-transit and late |
| | leakage beyond borders of treated area |

Cvnomolqus Monkey whole blood eotaxin-stimulated eosinophil shape change assay

Blood (10 mL) was taken from Cynomolgus monkeys and 1.1 ml of 3.8% sodium citrate solution added. Aliquots (90 μl) were incubated with antagonist at room temperature for 10 minutes then transferred to BSA-coated micronic tubes containing agonist (10 μl human eotaxin (Peprotech) Eotaxin was present at final concentrations between 0 and 1000 nM. The cells were incubated at 37° C. for a further 4 minutes before addition of 250 ml of ice-cold fixation buffer (a ¼ dilution of 1x Cellfix (Becton Dickinson)). After a minimum of 2 minutes the fixed samples were transferred into 2 ml of ice cold erythrocyte lysis buffer (155 mM NH₄Cl, 10 mM KHCO₃) and incubated on ice until lysis was complete (˜30 minutes). The mean forward scatter of the eosinophil population was then determined on a FACScalibur flow cytometer.

Oxygen-Induced Retinopathy

C57BL/6 mice placed in 75% O₂ at postnatal day (P) 7 and at P12 were returned to room air and treatment initiated with compound or vehicle. At P17, the area of retinal NV on the surface of the retina was measured as described previously (Shen et al., 2007). Briefly, P17 mice were given an intraocular injection of 1 μl of rat anti-mouse platelet endothelial cell adhesion molecule-1 (PECAM-1) antibody (Pharmingen, San Jose, Calif., USA); after 12 h, they were euthanized, and eyes were fixed in PBS-buffered formalin for 5 h at room temperature. Retinas were dissected, washed, and incubated with goat-anti rat polyclonal antibody conjugated with Alexa 488 (Invitrogen, Carlsbad, Calif., USA) at 1:500 dilution at room temperature for 45 min and flat mounted. An observer masked with respect to treatment group measured the area of NV per retina by image analysis.

Observations

Evaluation of the CCR3 inhibitor '994 in the Mouse Laser CNV Model

Systemic CCR3 antagonism with CCR3 inhibitor '994 limits the development of CNV in mice secondary to laser-induced photocoaqulation

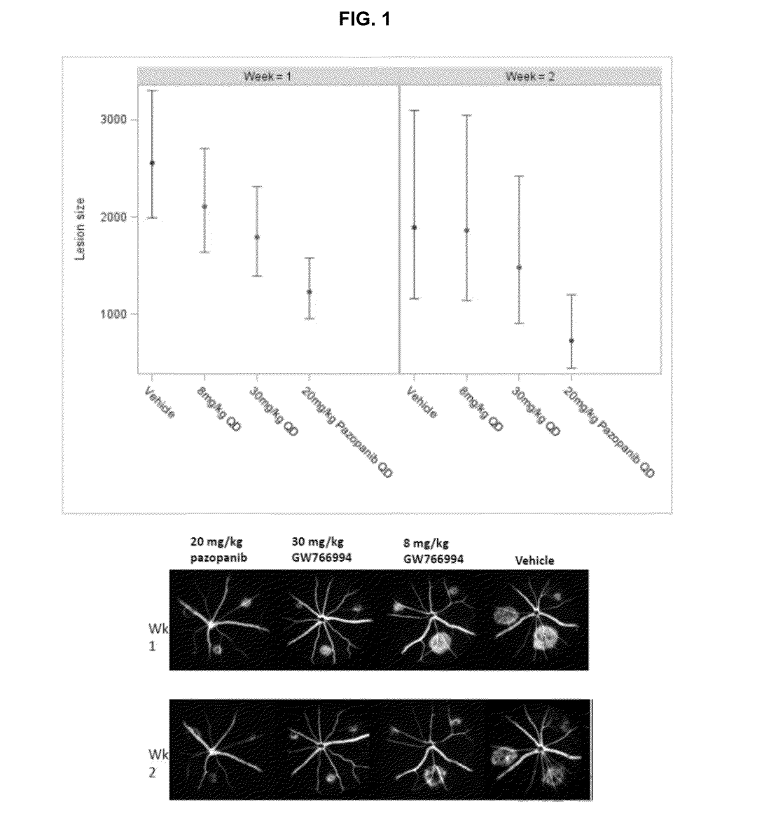

Adult 12 weeks old female C57BL/6 mice were used to generate the laser-induced CNV (3 CNV lesions per mouse retina) model using laser photocoagulation and puncture of Bruch's membrane. Studies in these mice in which CCR3 inhibitor '994 was orally dosed on the day of CNV induction by laser-photocoagulation showed a significant trend to effect at the 30 mg po QD dose (p=0.0525) at week 1 which was not sustained at week 2 (see, FIG. 1, upper and lower panels) and Table 1.

TABLE 1
Statistical comparisons for treatment effect of CCR3 inhibitor ′994
and pazopanib versus vehicle on laser-induced CNV lesion size
area in C57BI/6 mice as assessed by fluorescence angiography

| | Week 1 | | Week 2 | |
|---|---|---|---|---|
| | Ratio of | | Ratio of | |
| | mean lesion | | mean lesion | |
| Comparison | sizes | P-value | sizes | P-value |
| 8 mg/kg v Vehicle | 0.82 | 0.2721 | 0.99 | 0.9687 |
| 30 mg/kg v Vehicle | 0.70 | 0.0523 | 0.78 | 0.4825 |
| 20 mg/kg Pazopanib | 0.48 | 0.0001 | 0.39 | 0.0086 |
| v Vehicle | | | | |
| 30 mg/kg v 20 mg/kg | 1.47 | 0.0357 | 2.03 | 0.0455 |
| Pazopanib | | | | |

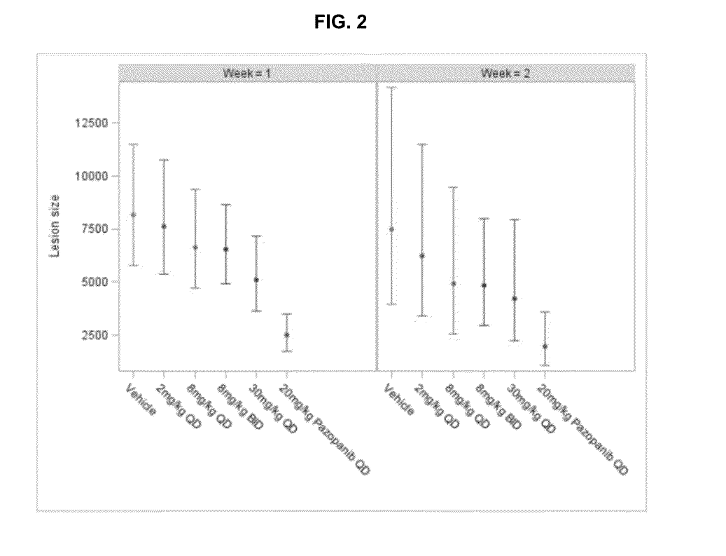

A similar effect was also observed in a second study at an identical dose (p=0.0552) (see, FIG. 2) and Table 2.

TABLE 2
Statistical comparisons for treatment effect of CCR3 inhibitor ′994
and pazopanib versus vehicle on laser-induced CNV lesion size
area in C57BI/6 mice as assessed by fluorescence angiography

| | Week 1 | | Week 2 | |
|---|---|---|---|---|
| | Ratio of | | Ratio of | |
| | mean lesion | | mean lesion | |
| Comparison | sizes | P-value | sizes | P-value |
| 2 mg/kg v Vehicle | 0.93 | 0.7780 | 0.83 | 0.6799 |
| 8 mg/kg v Vehicle | 0.81 | 0.4017 | 0.66 | 0.3582 |
| 8 mg/kg BID v Vehicle | 0.80 | 0.3174 | 0.65 | 0.2818 |
| 30 mg/kg v Vehicle | 0.62 | 0.0552 | 0.56 | 0.2036 |
| 20 mg/kg Pazopanib | 0.30 | <0.0001 | 0.26 | 0.0036 |
| v Vehicle | | | | |
| 30 mg/kg v 20 mg/kg | 2.05 | 0.0041 | 2.16 | 0.0837 |
| Pazopanib | | | | |

In both studies dosing of a comparator pazopanib at 20 mg/kg po QD, a spectrum specific kinase inhibitor active against VEGFR1, 2 and 3 (as well as PDGF receptors and c-kit), led to a significant reduction in CNV lesion size which was different from vehicle and different from all doses of CCR3 inhibitor '994 examined. The statistical analysis conducted here is stringent in that number of observations are related to the number of animals treated whereas in most other studies (conducted in the wider literature) using this laser-induced model each lesion (3 CNV lesions per mouse retina) are regarded as independent observations. If the data is analysed with each CNV lesion being regarded as independent then the effect of 30 mg/kg CCR3 inhibitor '994 in both studies would have been deemed to have had a significant effect on lesion size with p values of 0.0395 and 0.0392 respectively. However it is clear that in the case of orally dosed pazopanib either statistical approach would have yielded a robust effect or as such the effect of CCR3 inhibitor '994 in this model appears weaker than orally dosed pazopanib.

Evaluation of CCR3 inhibitor '994 in the Mouse JR5558 CNV Model

Systemic CCR3 antagonism with CCR3 inhibitor '994 limits the development of CNV in the JR5558 spontaneous model of CNV AMD

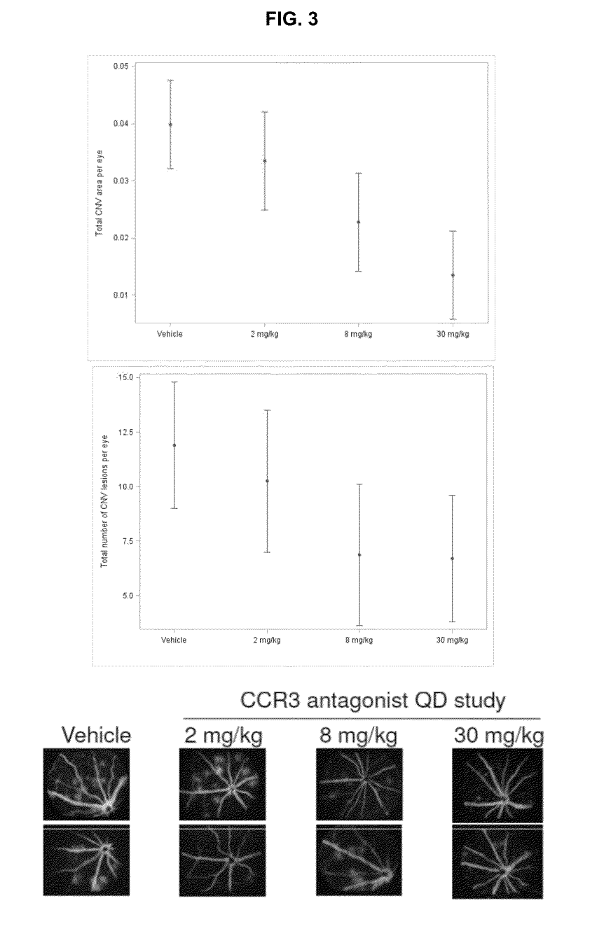

JR5558 mice are a strain of mice with an unidentified genetic defect which causes them to develop spontaneous CNV in both retinae with a predictable time course with the first lesions initiating at about post-natal day 12. As such treatment with CCR3 inhibitor '994 was initiated at day 12 and continued for 12 days at which point animals were examined by retinal fluorescence angiography to quantitate the total CNV lesion load and the total number of CNV lesions present in the retinae. Treatment of animals with CCR3 inhibitor '994 led to suppression of both total lesion area (see, FIG. 3, Table 3) and in the total number of CNV lesions present in the retinae (see, FIG. 3, Table 4) when dosed i.p. at either 8 mg/kg QD or 30 mg/kg QD but not when the dose was 2 mg/kg QD. Statistical values are shown in Table 3 and Table 4.

TABLE 3
Statistical comparisons for treatment effect of GW766994
versus vehicle on total CNV area in JR8885 study as assessed
by fluorescence angiography. Compound was
dosed for 12 days between P14 and P26

| | Difference in mean | |
|---|---|---|
| Comparison | total CNV area per eye | P-value |
| 2 mg/kg v Vehicle | −0.0065 | 0.2510 |
| 8 mg/kg v Vehicle | −0.0171 | 0.0066 |
| 30 mg/kg v Vehicle | −0.0264 | 0.0001 |

TABLE 4
Statistical comparisons for treatment effect of GW766994 versus
vehicle on total CNV number in JR8885 study as assessed by
fluorescence angiography. Compound was dosed for
12 days between P14 and P26

| | Difference in mean total | |
|---|---|---|
| Comparison | number of CNV lesions per eye | P-value |
| 2 mg/kg v Vehicle | −1.65 | 0.4312 |
| 8 mg/kg v Vehicle | −5.03 | 0.0271 |
| 30 mg/kg v Vehicle | −5.20 | 0.0169 |

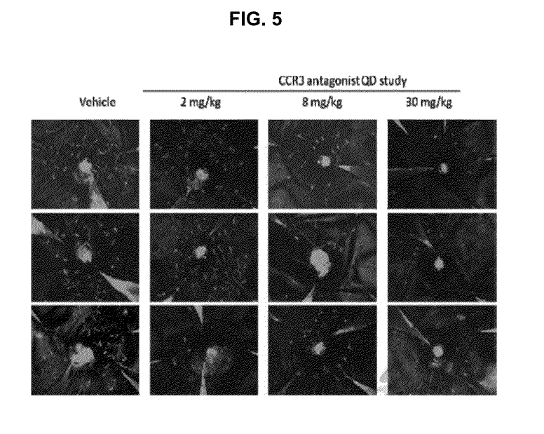

Immunohistochemistry of isolated eye cups (retina removed) also shows the effect of CCR3 inhibitor '994 in limiting the development of retinal vascular lesions in this model (FIG. 5). Although the 8 mg/kg QD dose of CCR3 inhibitor '994 did not show a statistically robust effect in the laser CNV model, in that model the compound was dosed orally which provides a 5-fold reduction in exposure as compared to i.p dosing which was that utilised in the JR8885 juvenile mouse model.

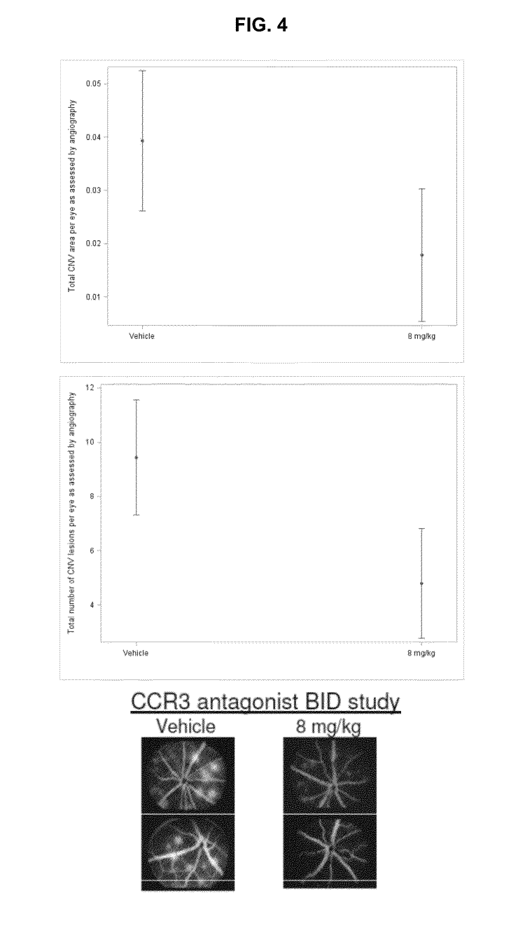

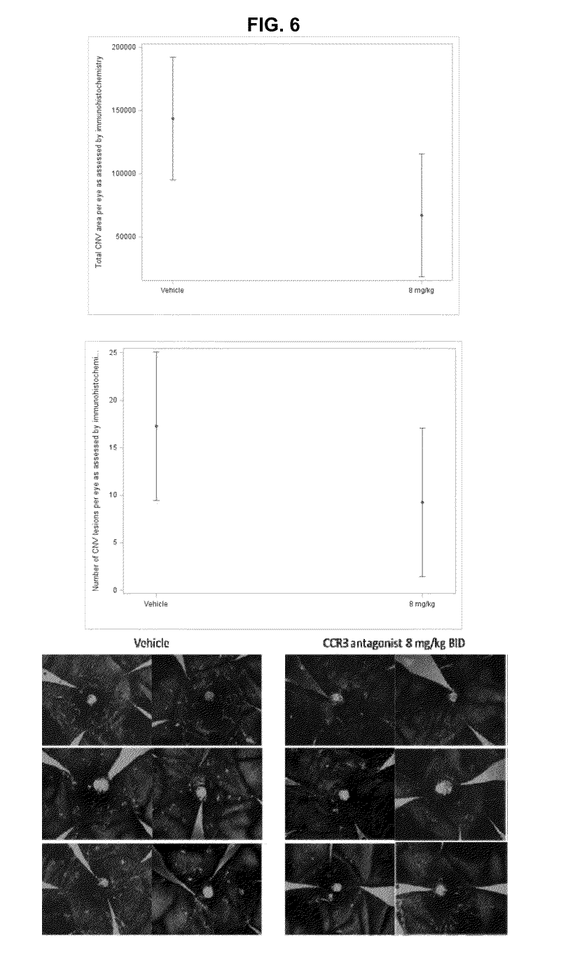

When CCR3 inhibitor '994 was dosed BID instead of QD, CCR3 inhibitor '994 also produced a significant effect in limiting both total lesion (see, FIG. 4, Table 5) area and total CNV number (see, FIG. 4, Table 6). In this study, animals were sacrificed immediately after the completion of fluorescence angiography and the eyes were removed and placed in fixative. Eyes were completely washed and then analysed by quantitative immunohistochemistry. Such analysis demonstrated that this method of analysis correlated well with live fluorescence angiography in showing a treatment effect of 8 mg/kg CCR3 inhibitor '994 BID on the lesion load but did not correlate with CNV lesion number as assessed by fluorescence angiography in this mouse model (see, FIG. 6) perhaps indicating that some of the lesions were either smaller and easier to detect by fluorescence angiography. Statistical values are shown in Tables 5 through 8.

TABLE 5
Statistical comparisons for treatment effect of CCR3 inhibitor
′994 versus vehicle on total CNV area in JR8885 study as
assessed by fluorescence angiography. Compound was
dosed for 12 days between P14 and P26

| | Difference in mean | |
|---|---|---|
| Comparison | total CNV area per eye | P-value |
| 8 mg/kg v Vehicle | −0.0214 | 0.0244 |

TABLE 6
Statistical comparisons for treatment effect of CCR3 inhibitor
′994 versus vehicle on total CNV number in JR8885 study
as assessed by fluorescence angiography. Compound was
dosed for 12 days between P14 and P26

| | Difference in mean number of | |
|---|---|---|
| Comparison | CNV lesions per eye | P-value |
| 8 mg/kg v Vehicle | −4.63 | 0.0049 |

TABLE 7
Statistical comparisons for treatment effect of CCR3 inhibitor
′994 versus vehicle on total CNV area in JR8885 study as
assessed by quantitative immunohistochemistry. Compound
was dosed for 12 days between P14 and P26

| | Difference in mean | |
|---|---|---|
| Comparison | total CNV area per eye | P-value |
| 8 mg/kg v Vehicle | −76632 | 0.0345 |

TABLE 8
Statistical comparisons for treatment effect of CCR3 inhibitor
′994 versus vehicle on total CNV number in JR8885 study
as assessed by quantitative immunohistochemistry. Compound
was dosed for 12 days between P14 and P26

| | Difference in mean number of | |
|---|---|---|
| Comparison | CNV lesions per eye | P-value |
| 8 mg/kg v Vehicle | −8.00 | 0.1280 |

Evaluation of CCR3 inhibitor '994 eye drops in the Mouse JR5558 CNV Model

CCR3 antagonism can limit CNV AMD after being applied topically in the mouse spontaneous JR5558 model

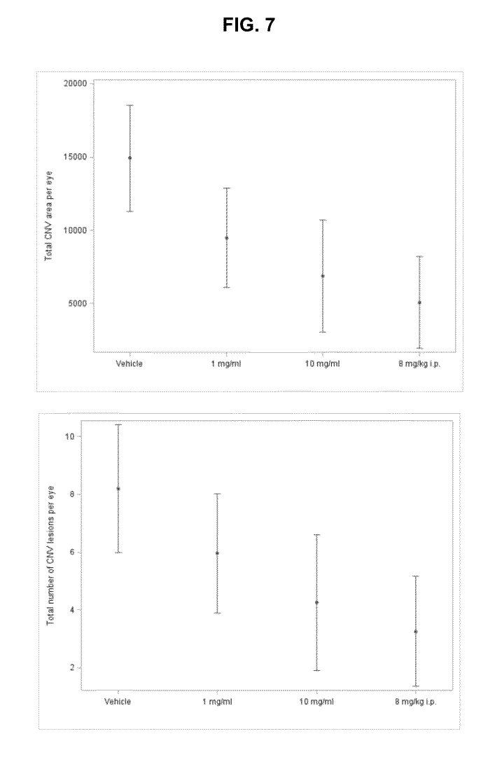

Dosing of CCR3 inhibitor '994 as a topically applied 5 ul eye drop to both eyes BID to JR5558 mice led to a statistically significant reduction in both total lesion load (see, FIG. 7, Table 9) and total number of spontaneous CNVs present in the retinae (see, FIG. 7, Table 10) of J5558 mice. The effect size was of similar magnitude as that obtained following systemic dosing of 8 mg/kg BID CCR3 inhibitor '994 which we had previously shown to be effective in limiting lesion load and total lesions in this model. The eye drop dosing regimen demonstrates that the effect of CCR3 inhibitor '994 on control CNV in this model is likely to be a local effect in view of the fact in previous experiments it was not possible to demonstrate efficacy in this model with a systemic dose of 2 mg/kg QD which is a greater mass of drug. Statistical values are shown in Table 9 and Table 10.

TABLE 9
Statistical comparisons for treatment effect of CCR3 inhibitor
′994 (dosed as 5 ul eye drop to each eye BID) versus vehicle
(dosed as 5 ul eye drop to each eye BID) on total CNV area in
JR8885 study as assessed by fluorescence angiography. 8 mg/kg
CCR3 inhibitor ′994 i.p. BID was also dosed as a comparator.
Compound was dosed for 12 days between P14 and P26

| | Difference in mean | |
|---|---|---|
| Comparison | total CNV area per eye | P-value |
| 1 mg/ml v Vehicle | −5442.63 | 0.0198 |
| 10 mg/ml v Vehicle | −8047.55 | 0.0010 |
| 8 mg/kg i.p v Vehicle | −9853.26 | 0.0002 |

TABLE 10
Statistical comparisons for treatment effect of CCR3 inhibitor
′994 (dosed as 5 ul eye drop to each eye BID) versus vehicle
(dosed as 5 ul eye drop to each eye BID) on total CNV number in
JR8885 study as assessed by fluorescence angiography. 8 mg/kg
CCR3 inhibitor ′994i.p. BID was also dosed as a comparator.
Compound was dosed for 12 days between P14 and P26

| | Difference in mean total | |
|---|---|---|
| Comparison | number of CNV lesions per eye | P-value |
| 1 mg/ml v Vehicle | −2.25 | 0.1094 |
| 10 mg/ml v Vehicle | −3.96 | 0.0071 |
| 8 mg/kg i.p. v Vehicle | 4.95 | 0.0017 |

Evaluation of CCR3 inhibitor '994 and an anti-VEGF Ab in the Mouse JR5558 CNV Model

CCR3 antagonism and inhibition of the VEGF pathway acts additively to limit the extent of CNV in the JR5558 spontaneous model

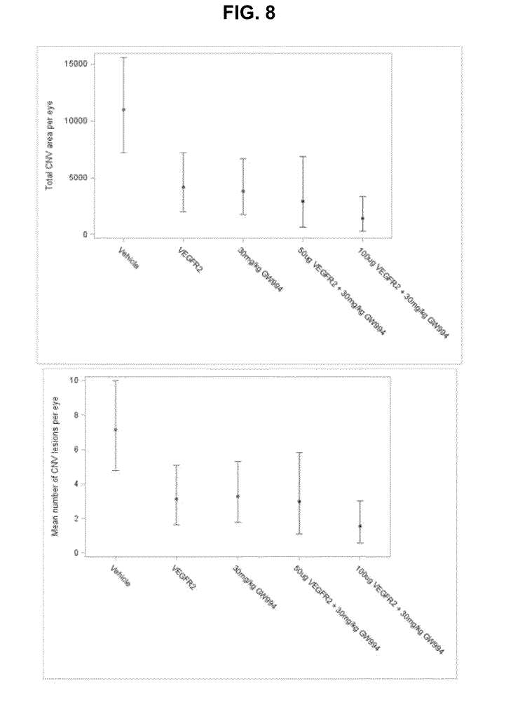

In experiments similar to those previous disclosed except that in this model systemic treatment with 30 mg/kg QD CCR3 inhibitor '994 led to a significant reduction in both CNV lesion area and total occurring CNV lesions when compared to vehicle (see, FIG. 8, Table 11 and Table 12). This profile was matched by systemic treatment with 50-10 ug anti-Vascular Endothelial Growth Factor Receptor-1 monoclonal antibody (anti-VEGFR2) (see, FIG. 8, Table 11 and Table 12). Combinations of 50-100 ug anti-VEGFR2 i.p. QD plus 30 mg CCR3 inhibitor '994 i.p. QD as expected also led to a suppression of both CNV lesion area and the number of CNV lesions. When examining the combination effect in comparison to the effect of single treatments with 30 mg/kg CCR3 inhibitor'994 i.p. QD or 100 ug anti-VEGFR2 i.p. QD there was further reductions in both the CNV lesion area and total number of lesions. These differences although not statistically significant at the 95% confidence limit showed a strong trend when comparing the combination with anti-VEGFR2 QD alone (p=0.06) (see, FIG. 8, Table 11) or 30 mg/kg CCR3 inhibitor '994 QD (p=0.09) (see, FIG. 8, Table 11) when examining the effects on the CNV lesion area. More moderate effects of the combination were evident when examining the effects of the combination on CNV lesion number versus single treatments alone (see, FIG. 8, Table 12).

TABLE 11
Statistical comparisons for treatment effect of 100 ug anti-
VEGFR2 i.p. QD, 30 mg/kg CCR3 inhibitor ′994 i.p. QD,
100 ug anti-VEGFR2 i.p. QD plus 30 mg/kg CCR3 inhibitor
′994 i.p QD and 100 ug anti-VEGFR2 i.p. QD plus 30 mg/kg
CCR3 inhibitor ′994 i.p. QD versus vehicle and versus
100 ug VEGFR2 i.p. QD and 30 mg/kg CCR3 inhibitor ′994 i.p.
QD on total CNV lesion area per retina in JR8885 study as
assessed by fluorescence angiography. CCR3 inhibitor ′994
was dosed for 12 days between P14 and P26. Anti-VEGFR2
was dosed for 6 days from P14 and a further 5 days from P19

| | | Adjusted |
|---|---|---|
| | | P-value |
| | | (Holm's |
| Comparison | P-value | correction) |
| 100 ug VEGFR2 v Vehicle | 0.0063 | 0.0379 |
| 30 mg/kg GW766994X v Vehicle | 0.0036 | 0.0284 |
| 50 ug anti-VEGFR2 + 30 mg/kg | 0.0052 | 0.0362 |
| GW766994X v Vehicle | | |
| 100 ug anti-VEGFR2 + 30 mg/kg | <0.0001 | 0.0002 |
| GW766994X v Vehicle | | |
| 50 ug anti-VEGFR2 + 30 mg/kg | 0.5528 | 0.9999 |
| GW766994X v 100 ug VEGFR2 | | |
| 50 ug anti-VEGFR2 + 30 mg/kg | 0.6714 | 0.9999 |
| GW766994X v 30 mg/kg GW766994X | | |
| 100 ug anti-VEGFR2 + 30 mg/kg | 0.0606 | 0.3032 |
| GW766994X v 100 ug VEGFR2 | | |
| 100 ug anti-VEGFR2 + 30 mg/kg | 0.0934 | 0.3736 |
| GW766994X v 30 mg/kg GW766994X | | |
| 100 ug anti-VEGFR2 + 30 mg/kg | 0.3367 | 0.9999 |
| GW766994X v 50 ug anti-VEGFR2 + 30 mg/kg | | |
| GW766994X | | |

TABLE 12
Statistical comparisons for treatment effect of 100 ug anti-
VEGFR2 i.p. QD, 30 mg/kg CCR3 inhibitor ′994 i.p. QD,
50 ug anti-VEGFR2 i.p. QD plus 30 mg/kg CCR3 inhibitor
′994 i.p QD and 50 ug anti-VEGFR2 i.p. QD plus 30 mg/kg
CCR3 inhibitor ′994 i.p. QD versus vehicle and versus
100 ug VEGFR2 i.p. QD and 30 mg/kg CCR3 inhibitor ′994 i.p.
QD on total CNV number in JR8885 study as assessed by
fluorescence angiography. CCR3 inhibitor ′994 was
dosed for 12 days between P14 and P26. Anti-VEGFR2 was
dosed for 6 days from P14 and a further 5 days from P19

| | | Adjusted |
|---|---|---|
| | | P-value |
| | | (Holm's |
| Comparison | P-value | correction) |
| 100 ug VEGFR2 v Vehicle | 0.0107 | 0.0860 |
| 30 mg/kg GW766994X v Vehicle | 0.0152 | 0.1066 |
| 50 ug anti-VEGFR2 + 30 mg/kg | 0.0283 | 0.1700 |
| GW766994X v Vehicle | | |
| 100 ug anti-VEGFR2 + 30 mg/kg | 0.0001 | 0.0011 |
| GW766994X v Vehicle | | |
| 50 ug anti-VEGFR2 + 30 mg/kg | 0.9250 | 0.9999 |
| GW766994X v 100 ug VEGFR2 | | |
| 50 ug anti-VEGFR2 + 30 mg/kg | 0.8374 | 0.9999 |
| GW766994X v 30 mg/kg GW766994X | | |
| 100 ug anti-VEGFR2 + 30 mg/kg | 0.1336 | 0.5345 |
| GW766994X v 100 ug VEGFR2 | | |
| 100 ug anti-VEGFR2 + 30 mg/kg | 0.1029 | 0.5144 |
| GW766994X v 30 mg/kg GW766994X | | |
| 100 ug anti-VEGFR2 + 30 mg/kg | 0.2554 | 0.7663 |
| GW766994X v 50 ug anti-VEGFR2 + 30 mg/kg | | |
| GW766994X | | |

Evaluation of CCR3 inhibitor '994 on permeability of CNV lesions in the Mouse JR5558CNV Model

CCR3 antagonism limits the vascular permeability of choroidal neovessels to sodium fluorescein in JR5558 mice.

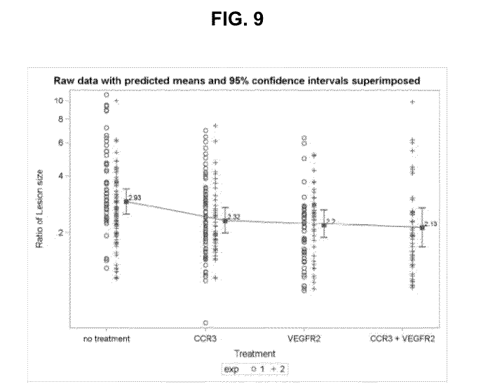

Previous literature reports have taught that CCR3 can mediate changes in the permeability of cultures of vascular endothelial cell (Jamaluddin et al., 2009). This, however has not to date been demonstrated on choroidal or retinal endothelium or indeed in vivo. Determination of changes in permeability of CNV lesions is calculated by subtracting late phase fluorescein leakage from early phase fluorescein leakage to determine a rate of leakage. The permeability is then calculated using these values for each individual CNV lesion. In contrast to experiments aimed at quantifying total CNV lesion load and total number of lesions, the action of compounds on permeability is determined following only 2 days (from P24-P26) dosing so CNV lesions have had the required time to develop and were not inhibited from developing with prior exposure to a CCR3 antagonist. Dosing of both CCR3 inhibitor '994 (30 mg/kg i.p QD. P=0.0351) and Anti-VEGF (100 ug i.p. QD, p=0.0176) both led to an independently statistically robust effect on reducing the vascular permeability of the CNV lesions to fluorescein. Combination of anti-VEGFR2 therapy with CCR3 inhibitor '994 led to a further reduction in CNV lesion vascular permeability which was significant from vehicle (p=0.0251, FIG. 9, table 13) but was not significantly different when compared to either CCR3 inhibitor '994 or anti-VEGFR2 mAb) when dosed alone (see, FIG. 9, Table 13)

TABLE 13
Statistical comparisons for treatment effect of 100 ug anti-
VEGFR2 i.p. QD, 30 mg/kg CCR3 inhibitor ′994 i.p. QD,
100 ug anti-VEGFR2 i.p. QD plus 30 mg/kg CCR3
inhibitor ′994. versus vehicle and versus 100 ug VEGFR2
i.p. QD and 30 mg/kg CCR3 inhibitor ′994 i.p. QD on the
permeability of CNV lesions in JR8885 study as assessed by
fluorescence angiography (late-early FFA for each lesion).
CCR3 inhibitor ′994 and anti-VEGFR2 were dosed
singularly or in combination for 2 days between P24 and P26

| | | Ratio of change with 95% |
|---|---|---|
| | p-value | confidence interval |
| Comparison with no | | |
| treatment | | |
| CCR3 | 0.0345 | 1.26 [1.02, 1.56) |
| Anti-VEGFR2 | 0.0176 | 1.31 [1.05, 1.04) |
| Comparison | | |
| CCR3 vs combined | 0.5291 | 1.09 [0.82, 1.45) |
| Anti-VEGFR2 vs combined | 0.7408 | 1.05 [0.79, 1.40) |
| Combined vs control | 0.0251 | 1.38 [1.04, 1.82) |

Evaluation of the '415 compound in the Cyno Laser CNV Model

Oral dosing of a CCR3 antagonist GW782415X limits laser-induced choroidal CNV in Cynomolgus monkey

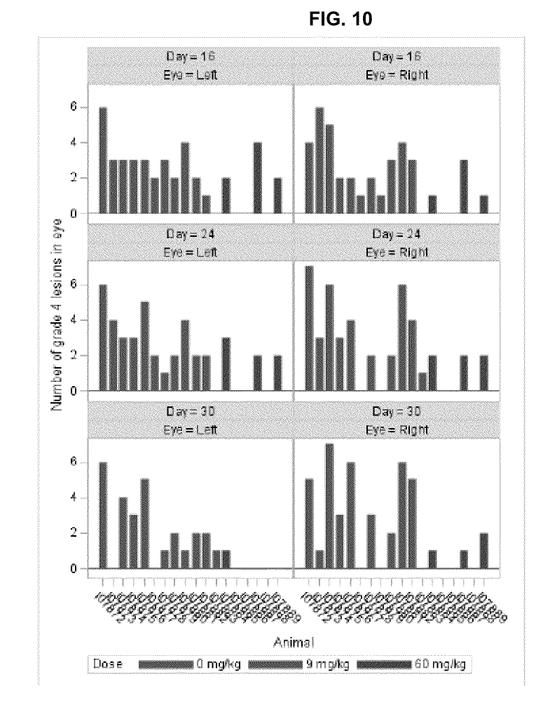

To determine if CCR3 antagonists would suppress the induction of CNV in higher mammals, a CCR3 antagonist tool molecule the '415 compound was chosen since it has good potency against Cynomolgus monkey CCR3 (pA₂=7-8 depending on experiment) and showed reasonable exposure in this primate with low inter-animal variability. The effect of dosing 20 mg/kg the '415 compound TID po for 15 days in this model demonstrated a statistically robust effect on the generation of grade IV lesions in this model at all time points (see, FIG. 10, Table 14), when assessed by fluorescence angiography at 16 , 24 and 30 days post-laser. The effect of a lower dose of the '415 compound showed a reduction in the number of lesions but this was not statistically different from controls given the power of the study (see, FIG. 10, Table 14). However the suppression of grade IV lesions by the lower 9 mg/kg the '415 compound TID dose was sufficient to demonstrate along with the 20 mg/kg the '415 compound dose that there was a statistically significant dose-relationship with regards to the suppression of grade IV CNV in this model at all three time-points studied, and this was independent of log dose scaling or linear dose scaling assumptions (see, Table 15)

TABLE 14
Statistical comparisons for treatment effect of the ′415
compound at 20 mg/kg po TID and 3 mg/kg po TID versus
vehicle on Grade IV CNV lesions in Cynomolgus monkey
assessed by fluorescence angiography at days 16, 24 and 30

| | | Adjusted P-value |
|---|---|---|
| Comparison | P-value | (Holm's correction) |
| 9 mg/kg v Vehicle at day 16 | 0.0819 | 0.5129 |
| 9 mg/kg v Vehicle at day 24 | 0.0781 | 0.5129 |
| 9 mg/kg v Vehicle at day 30 | 0.3913 | 0.8694 |
| 60 mg/kg v Vehcle at day 16 | 0.0005 | 0.0070 |
| 60 mg/kg v Vehicle at day 24 | 0.0009 | 0.0112 |
| 60 mg/kg v Vehicle at day 30 | 0.0011 | 0.0121 |

TABLE 15
Statistical comparisons for treatment effect and dose-effect
relationship for the ′415 compound dosed at 20 mg/kg po
TID and 3 mg/kg po TID versus vehicle on Grade IV CNV
lesions in Cynomolgus monkey assessed by fluorescence
angiography at days 16, 24 and 30

| | | Adjusted P-value |
|---|---|---|
| Time point | P-value | (Holm's correction) |
| Day 16 | 0.0005 | 0.0070 |
| Day 24 | 0.0009 | 0.0112 |
| Day 30 | 0.0014 | 0.0139 |

Evaluation of the effect of systemic and topical CCR3 inhibitor '994 on Retinal neovascularization induced by hyperbaric oxygen (Oxygen-Induced Retinopathy)

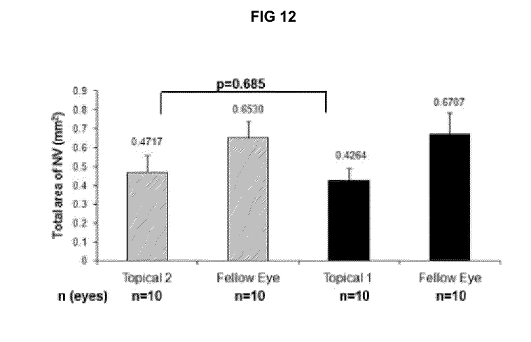

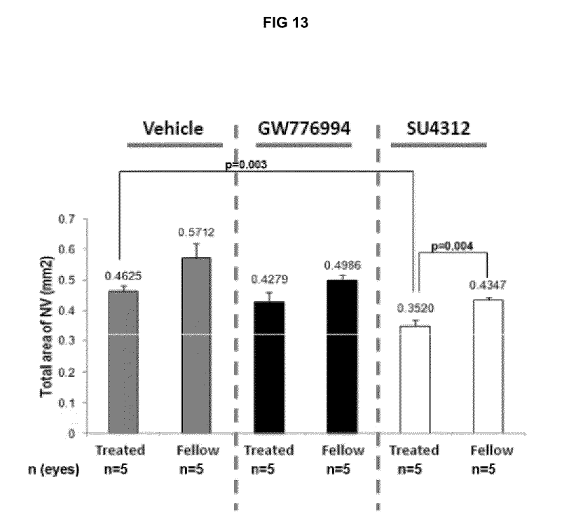

Treatment of C57B16 mice with CCR3 inhibitor '994 either topically (5 ul 10 mg/ml BID) or via systemic administration 8 mg/kg BID i.p. at doses which were effective in models of choroidal neovascularization did not have an effect on the neovascularization of retinal vessels following stimulation with hyperbaric oxygen. (see, FIGS. 12 and 13). In systemic experiments both eyes were examined and no significant differences were observed between CCR3 inhibitor '994 and vehicle treated groups. In topical experiments no significant differences were noted between CCR3 inhibitor '994 and vehicle groups nor treated and fellow eye in the CCR3 inhibitor '994 treated group. It is notable that the receptor tyrosine kinase inhibitor SU4312 at a dose of 5 ug (dosed peri-ocularly every 5 days) was an effective inhibitor and significantly observable reductions in retinal neovascularization were observed between SU4312 and vehicle treated eyes (p<0.003) and indeed between treated and fellow (non-treated) eyes (p<0.004). Consequently SU4312 acts similarly on retinal neovascularization to that of an alternative receptor tyrosine kinase inhibitor pazopanib which was effective in limiting choroidal neovascularization. This data demonstrates that blockade of receptor tyrosine kinase (e.g., VEGF) is pertinent to both retinal and choroidal neovascularization whereas CCR3 antagonism seems to only limit choroidal neovascularization thus highlighting the fact that CCR3 antagonism is selective to the choroid and consequently not a generally applicable mechanism which can be applied to all vascular beds, even within the eye.

PK/PD for CNV AMD in rodent and primate models

CCR3 inhibitor '994 and development of spontaneous CNV in the JR5558 mouse model

Following dosing of 8 mg/kg i.p QD CCR3 inhibitor '994 in juvenile mice to mimic exposure effects in JR5558 mice, blood concentration at 24 hrs was calculated for 2 mg/kg i.p, 8 mg.kg i.p and 30 mg/kg i.p. doses by projecting the log linear phase of the PK profile. Blood drug concentrations were then used to determine fractional receptor occupancy using the Langmuir binding isotherm and the pKi for CCR3 inhibitor '994 (estimated in a mouse eosinophil chemotaxis assay). The receptor occupancy values are small over estimates of the true value as the pKi for CCR3 inhibitor '994 was determined in buffer and not blood. The fractional CCR3 receptor occupancy will largely be due to binding to eosinophils as this is the predominant CCR3 bearing cell population. The calculations of fractional receptor occupancy imply that at 8 hours all doses had sufficient exposure to result in greater than 97% fractional CCR3 receptor occupancy (2 mg/kg=97.95%, 8 mg/kg=99.48%, 30 mg/kg=99.86%) but at the 24 h trough time point fractional CCR3 receptor occupancy values were different (2 mg/kg=3.1%, 8 mg/kg=11.36, 30 mg/kg=32.45). Since systemic dosing of both 8 mg/kg and 30 mg/kg CCR3 inhibitor '994 was efficacious in the model it would appear that systemic fractional occupancy levels at trough between 3.1% and 11.36% are required for efficacy. However, a number of observations suggest that the impact of CCR3 inhibitor '994 on the generation of CNV in the JR5558 model is not driven by overall fractional CCR3 receptor occupancy or that a systemic effect of CCR3 inhibitor '994 in mediating CNV in this model. Firstly, Takeda et al., 2009 demonstrated an effect of CCR3 inhibitors in moderating laser-induced CNV in animals which were genetically incapable of generating either eosinophils or mast cells (the major CCR3 bearing cells). This same group also used very small doses of anti-CCR3 antibodies dosed by intravitreal injection to limit laser-induced CNV in mouse models and these very small doses would have very limited impact of fractional receptor occupancy even if they were able to access the systemic circulation. In addition, topical dosing of CCR3 inhibitor '994 as an eye drop formulation was also efficacious in the JR5558 model. All these observations taken together suggest that the effect of CCR3 therapeutics in limiting CNV disease is via a local activity in the eye. It is highly likely however that systemic dosing drives exposure of CCR3 antagonist in the eye and ultimately the effect on disease and the exposure in the eye is likely to be a complex function of the systemic exposure and thus an understanding of the systemic pharmacokinetics driving efficacy may be important in profiling systemic therapies.

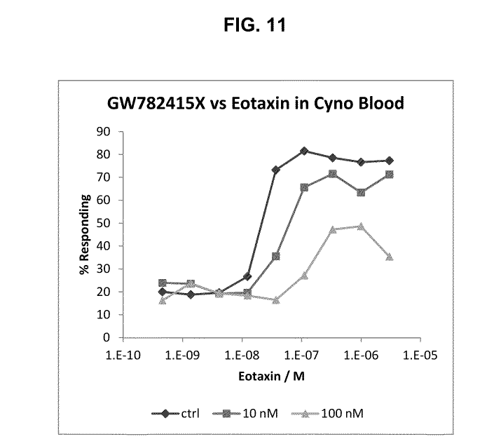

The '415 compound caused a concentration-dependent, surmountable inhibition of human eotaxin-induced eosinophil shape change in Cynomolgus whole blood. Schild analysis of these data gave a mean pA₂ of between 7.8-8,4 for the '415 compound. The concentration response curve of eotaxin-1 on eosinophil shape change in Cynomolgus whole blood and the effect of pre-incubation of 10 nM and 100 nM the '415 compound is shown in FIG. 11.

Using trough levels of exposure of the '415 compound at 8 hours for both the 9 mg/kg/day and the 60 mg/kg/day, as compound was dosed TID, and using potency values determined in the eotaxin-mediated eosinophil shape change assay in Cynomolgus whole blood CCR3, the fractional receptor occupancy was calculated to be >95% at trough.

Taken together, data is provided in three in vivo models which demonstrate a strong pharmacological effect of a CCR3 antagonists administered systemically or by local eye drop delivery on the development of choroidal neovascular lesions. In rodent models the effects of the administration of a CCR3 antagonist are similar in magnitude to the effects of a systemically administered anti-VEGR2 or orally dosed pazopanib. As well as an effect of the growth of CNV lesions we are also able to demonstrate that both anti-VEGFR2 and a CCR3 antagonist are effective in reducing the increase in vascular permeability to sodium fluoroscien which is a hallmark of the clinical disease in humans. Previous literature data also supports a local effect of both anti-VEGF strategies and a CCR3 antagonist approach in controlling CNV development (Takeda et al., 2009). The fact that local intravitreal delivery of small amounts of anti-CCR3 mAb or intravitreal administration of Mabs targeting the ligands of CCR3 and the fact that laser-induced CNV generation is still possible in mice genetically incapable of producing eosinophils or mast cells argues strongly that such CCR3 mechanism is local and not dependent on circulating eosinophils (Takeda et al., 2009). Such literature studies also suggested that the anti-VEGF and CCR3 mechanisms were independent based on the findings that control of CNV in rodents with anti-VEGFs had no impact on CCR3-bearing cells in the eye and studies using intravitreal anti-CCR3 mabs in laser-induced CNV had no impact on vitreal VEGF levels induced following laser photocoagulation (Takeda et al., 2009). Our data demonstrates that both CCR3 and VEGFR2 alone limit CNV lesion volume, lesion number and vascular permeability of CNV lesions, and whilst this has previously been disclosed for anti-VEGF therapeutics a specific effect on CNV number and vascular permeability has not been described for CCR3 antagonists. Additionally enhanced combination effect of the anti-VEGFR2 mAb and CCR3 antagonist approaches on CNV lesion volume, lesion number and vascular permeability of CNV lesions is demonstrated herein. It is also important to note the effect of CCR3 therapy alone and in combination with VEGF therapeutics on the number of emerging lesions in then spontaneous JR5558 model since these lesions arise on a background of atrophic retina which is similar to high incidence of CNV disease occurring in eyes with non-vascular AMD and geographic atrophy AMD. The presence of dry or geographic atrophic AMD confers significant risk on the likelihood of the eye transitioning to neovascular AMD. Specifically the JR5558 model is noted to show retinal atrophy, retinal cell apoptosis, and associated inflammation (data not shown) which are all hallmarks of non-vascular AMD in humans. It is unlikely that invasive treatments such as intravitreal injections would be acceptable as a treatment to prevent the risk of transitioning from non-vascular AMD to neovascular AMD but an oral treatment which would potentially protect both eyes could be envisioned using a systemically or topically applied CCR3 antagonist. The CCR3 antagonist approach represents a novel and specific mechanism in controlling both choroidal neovascularization and vascular permeability both of which are key mechanisms in the generation of CNV lesions and the weight of evidence suggests that the effects of anti-CCR3 therapeutics are local to the eye and that the effects are mediated through an independent mechanism of action to that mediated by VEGF. Although recent data has demonstrated that VEGF and CCR3 may activate similar signaling pathways in human choroidal endothelial cells such as the small GTPase rac1 and the effect of these two agents can be additive it has not yet been shown that simultaneous blockade of both CCR3 and VEGF pathways leads to a combination effect in suppressing CNV (Wang et al., 2011). Also we demonstrate that CCR3 is not active in suppressing retinal neovascuarisation induced by hyperbaric oxygen whereas anti-VEGF therapeutics is very effective in this model, pointing to the fact that there may be a tissue selectivity of the CCR3 mechanism. Studies which initially pointed to a general effect of CCR3 in mediating angiogenesis in a number of tissues (Salcedo et al., 2001) and vascular permeability in heart-derived endothelial cell cultures (Jamaluddin et al., 2009) are clearly not generally applicable to all tissues and indeed all tissues of the eyes and particularly sites of ocular angiogenesis and enhanced vascular permeability.

In some embodiments, the optimum dosage of agents that inhibit CCR3 is one that reduces activity and/or expression of CCR3, for example, reduced expression of nucleic acid, for example mRNA encoded by CCR3 gene or reduced expression or activity of CCR3 protein. In other embodiments, the optimum dosage of agents that inhibit CCR3 is one that generates the maximum protective effect in preventing an ocular disease or disorder including, for example, but not limited to, neovascular age-related macular edema and neovascular age-related macular edema secondary to dry or atrophic AMD.

Formulations of compositions

Compounds, for example agents inhibiting CCR3 as disclosed herein, can be used as a medicament or used to formulate a pharmaceutical composition with one or more of the utilities disclosed herein. They can be administered in vitro to cells in culture, in vivo to cells in the body, or ex vivo to cells outside of an individual that can later be returned to the body of the same individual or another. Such cells can be disaggregated or provided as solid tissue.

Compounds, for example agents inhibiting CCR3 as disclosed herein can be used to produce a medicament or other pharmaceutical compositions. Use of agents inhibiting CCR3 which further comprise a pharmaceutically acceptable carrier and compositions which further comprise components useful for delivering the composition to an individual are known in the art. Addition of such carriers and other components to the agents as disclosed herein is well within the level of skill in this art.

Pharmaceutical compositions can be administered as a formulation adapted for passage through the blood-brain barrier or direct contact with the endothelium. In some embodiments, the compositions may be administered as a formulation adapted for systemic delivery. In some embodiments, the compositions may be administered as a formulation adapted for delivery to specific organs, for example but not limited to the liver, bone marrow, or systemic delivery.

Alternatively, pharmaceutical compositions can be added to the culture medium of cells ex vivo. In addition to the active compound, such compositions can contain pharmaceutically-acceptable carriers and other ingredients known to facilitate administration and/or enhance uptake (e.g., saline, dimethyl sulfoxide, lipid, polymer, affinity-based cell specific-targeting systems). The composition can be incorporated in a gel, sponge, or other permeable matrix (e.g., formed as pellets or a disk) and placed in proximity to the endothelium for sustained, local release. The composition can be administered in a single dose or in multiple doses which are administered at different times.

Pharmaceutical compositions can be administered by any known route. By way of example, the composition can be administered by a mucosal, pulmonary, topical, or other localized or systemic route (e.g., enteral and parenteral). The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. The phrases “systemic administration,” “administered systemically”, “peripheral administration” and “administered peripherally” as used herein mean the administration of the agents as disclosed herein such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert.

Suitable choices in amounts and timing of doses, formulation, and routes of administration can be made with the goals of achieving a favorable response in the subject with diabetic ocular diseases or a risk thereof (i.e., efficacy), and avoiding undue toxicity or other harm thereto (i.e., safety). Therefore, “effective” refers to such choices that involve routine manipulation of conditions to achieve a desired effect.

A bolus of the formulation administered to an individual over a short time once a day is a convenient dosing schedule. Alternatively, the effective daily dose can be divided into multiple doses for purposes of administration, for example, two to twelve doses per day. Dosage levels of active ingredients in a pharmaceutical composition can also be varied so as to achieve a transient or sustained concentration of the compound or derivative thereof in an individual and to result in the desired therapeutic response or protection. But it is also within the skill of the art to start doses at levels lower than required to achieve the desired therapeutic effect and to gradually increase the dosage until the desired effect is achieved.

The amount of agents inhibiting CCR3 administered is dependent upon factors known to a person skilled in the art such as bioactivity and bioavailability of the compound (e.g., half-life in the body, stability, and metabolism); chemical properties of the compound (e.g., molecular weight, hydrophobicity, and solubility); route and scheduling of administration, and the like. It will also be understood that the specific dose level to be achieved for any particular individual can depend on a variety of factors, including age, gender, health, medical history, weight, combination with one or more other drugs, and severity of disease.

The term “treatment”, with respect to treatment of AMD refers to, inter alia, preventing the development of the disease, or altering the course of the disease (for example, but not limited to, slowing the progression of the disease), or reversing a symptom of the disease or reducing one or more symptoms and/or one or more biochemical markers in a subject, preventing one or more symptoms from worsening or progressing, promoting recovery or improving prognosis, and/or preventing disease in a subject who is free there from as well as slowing or reducing progression of existing disease. For a given subject, improvement in a symptom, its worsening, regression, or progression can be determined by an objective or subjective measure.

Prophylactic methods (e.g., preventing or reducing the incidence of relapse) are also considered treatment.

In some embodiments, treatment can also involve combination with other existing modes of treatment, for example existing agents for treatment of diabetic ocular diseases , such as anti VEGF therapeutics e.g. Lucentis®, Avastin®, and Aflibercept® and steroids, e.g., triamcinolone, and steroid implants containing fluocinolone acetonide.

Thus, combination treatment with one or more agents that inhibit CCR3 with one or more other medical procedures can be practiced.

In addition, treatment can also comprise multiple agents to inhibit CCR3 expression or activity.

Where a combination therapy is employed, the therapeutic agents may be administered together or separately. The same means for administration may be used for more than one therapeutic agent of the combination therapy; alternatively, different therapeutic agents of the combination therapy may be administered by different means. When the therapeutic agents are administered separately, they may be administered simultaneously or sequentially in any order, both close and remote in time. The amounts of the CCR3 inhibitory compound, and/or and the other pharmaceutically active agent or agents, e.g., an anti-VEGF therapeutic, and the relative timings of administration will be selected in order to achieve the desired combined therapeutic effect.

The amount which is administered to a subject is preferably an amount that does not induce toxic effects which outweigh the advantages which result from its administration. Further objectives are to reduce in number, diminish in severity, and/or otherwise relieve suffering from the symptoms of the disease in the individual in comparison to recognized standards of care.

Production of compounds according to present regulations will be regulated for good laboratory practices (GLP) and good manufacturing practices (GMP) by governmental agencies (e.g., U.S. Food and Drug Administration). This requires accurate and complete record keeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

Dosages, formulations, dosage volumes, regimens, and methods for analyzing results aimed at inhibiting CCR3 expression and/or activity can vary. Thus, minimum and maximum effective dosages vary depending on the method of administration. Suppression of the clinical and histological changes associated with AMD can occur within a specific dosage range, which, however, varies depending on the organism receiving the dosage, the route of administration, whether agents that inhibit CCR3 are administered in conjunction with other co-stimulatory molecules, and the specific regimen of inhibitor of CCR3 administration. For example, in general, nasal administration requires a smaller dosage than oral, enteral, rectal, or vaginal administration.

For oral or enteral formulations for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4, 556,552, “Enteric Film- Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982.

The treatment of AMD with CCR3 inhibitors may also be administered locally, as a topical eye drop, a peri-ocular injection (e.g., sub-tenon), via intravitreal injection, or using iontophoresis, peri-ocular devices which can actively or passively deliver drug. Sustained release of drug may also be achieved by the use of technologies such as solid implants (which may or may not be bio-degradable) or bio-degradable polymeric matrices (e.g. micro-particles). These may be administered either peri-ocularly or intravitreally.

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent. Formulations to be administered to the eye will have ophthalmically compatible pH and osmolality. One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodable polymers (e.g. poly(ethylene vinyl) acetate (EVA), superhydrolyzed PVA), hydroxyalkyl cellulose (HPC), methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition and polymeric coatings that will enhance drug diffusion, erosion, dissolution and osmosis.

Formulations for drug delivery using ocular devices may combine one or more active agents and adjuvants appropriate for the indicated route of administration. For example, the active agents may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers, and a carrier or diluent that has a time delay property. Representative examples of biodegradable compositions can include albumin, gelatin, starch, cellulose, dextrans, polysaccharides, poly (D,L-lactide), poly (D,L-lactide-co-glycolide), poly (glycolide), poly (hydroxybutyrate), poly (alkylcarbonate) and poly (orthoesters) and mixtures thereof. Representative examples of non-biodegradable polymers can include EVA copolymers, silicone rubber and poly (methylacrylate), and mixtures thereof.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein is includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig (2005) Adv. Drug Deliv. Rev. 3; 57:1595-639, herein incorporated by reference for purposes of its teachings of examples of polymers for use in ocular drug delivery.

### REFERENCES

The references cited herein and throughout the application are incorporated herein by reference.

Salcedo R, Young H A, Ponce M L, Ward J M, Kleinman H K, Murphy W J, Oppenheim J J. Eotaxin (CCL 11) induces in vivo angiogenic responses by human CCR3+endothelial cells, J Immunol., Jun 15; 166(12):7571-8 (2001). Erratum in: J Immunol Jan 1; 168(1):511 (2002).

Jamaluddin M S, Wang X, Wang H, Rafael C, Yao Q, Chen C. Eotaxin increases monolayer permeability of human coronary artery endothelial cells, Arterioscler Thromb Vasc Biol., Dec; 29(12):2146-52 (2009).

Mo F M, Proia A D, Johnson W H, Cyr D, Lashkari K. Interferon gamma-inducible protein-10 (IP-10) and eotaxin as biomarkers in age-related macular degeneration, Invest Ophthalmol Vis Sci., Aug; 51(8):4226-36 (2010). Epub 2010 Mar. 10, 2010.

Takeda A, Baffi J Z, Kleinman M E, Cho W G, Nozaki M, Yamada K, Kaneko H, Albuquerque R J, Dridi S, Saito K, Raisler B J, Budd S J, Geisen P, Munitz A, Ambati B K, Green M G, Ishibashi T, Wright J D, Humbles A A, Gerard C J, Ogura Y, Pan Y, Smith J R, Grisanti S, Hartnett M E, Rothenberg M E, Ambati J. CCR3 is a target for age-related macular degeneration diagnosis and therapy, Nature, Jul 9; 460(7252):225-30 (2009). Epub 2009 Jun. 14, 2009.

Mizutani et al., ARVO abstract 2011, A386

Li Y, Huang D, Xia X, Wang Z, Luo L, Wen R. CCR3 and choroidal neovascularization, PLoS One, Feb 15; 6(2):e17106 (2011).

Liclican E L, Nguyen V, Sullivan A B, Gronert K. Selective activation of the prostaglandin E2 circuit in chronic injury-induced pathologic angiogenesis, Invest Ophthalmol Vis Sci. Dec; 51(12):6311-20. Epub 2010 Jul. 7, 2010.

Sato T, Kusaka S, Shimojo H, Fujikado T; Simultaneous analyses of vitreous levels of 27 cytokines in eyes with retinopathy of prematurity, Ophthalmology, Nov; 116(11):2165-9. Epub 2009 Aug. 22, 2009.

Wang H, Wittchen E S, Jiang Y, Ambati B, Grossniklaus H E, Hartnett M E.2011 Upregulation of CCR3 by age-related stresses promotes choroidal endothelial cell migration via VEGF-dependent and independent signalling. Invest Ophthalmol Vis Sci. Sep 14

Shen, J., Xie, B., Dong, A., Swaim, M., Hackett, S. F., Campochiaro, P. A. (2007) In vivo immunostaining demonstrates macrophages associate with growing and regressing vessels. Invest. Ophthalmol. Vis. Sci. 48,4335-4341.

1. A method of treating and/or preventing an ocular disorder or disease associated with neovascularization in a subject, the method comprising identifying a subject with, or at risk of developing an ocular disease or disorder associated with neovascularization, and administering to the subject in need thereof a pharmaceutical composition comprising an agent for inhibiting the activity and/or expression of the CCR3 protein.

2. The method of claim 1, wherein the ocular disease or disorder is neovascular AMD.

3. A method of treating and/or preventing an ocular disorder or disease associated with increased choroidal vascular permeability in a subject, the method comprising identifying a subject with, or at risk of developing neovascular AMD or disorder associated with increased choroidal vascular permeability, and administering to the subject in need thereof a pharmaceutical composition comprising an agent for inhibiting the activity and/or expression of the CCR3 protein.

4. The method of claim 1, wherein the ocular disease or disorder is dry or geographic atrophy AMD.

5. A method of treating and/or preventing AMD in a subject with, or at risk of AMD, comprising administering to the subject a pharmaceutical composition comprising an agent which inhibits the activity and/or expression of CCR3 protein.

6. The method of claim 5, wherein inhibition of the CCR3 protein reduces or stops a symptom of AMD.

7. A method of treating and/or preventing AMD in a subject with, or at risk of AMD, comprising administering to the subject a pharmaceutical composition comprising an agent which inhibits the activity and/or expression of CCR3 protein, wherein inhibition of the CCR3 protein reduces or stops a symptom of AMD, and further administering in combination with the agent, an anti-VEGF inhibitor.

8. A method of preventing the development of a CNV lesion on an atrophic retinal background in a subject at risk of developing choroidal neovascuarisation and/or subsequent increased choroidal vascular permeability, comprising administering to the subject a pharmaceutical composition comprising an agent which inhibits the activity and/or expression of CCR3 protein, wherein inhibition of the CCR3 protein prevents the development of such lesion, which agent can be administered alone or in combination with an anti-VEGF inhibitor.

9. A method of preventing the transition of atrophic and non-vascular AMD to neovascular AMD comprising administering to the subject a pharmaceutical composition comprising an agent which inhibits the activity and/or expression of CCR3 protein, wherein inhibition of the CCR3 protein such transition to neovascular AMD, which agent can be administered alone or in combination with an anti-VEGF inhibitor.

10. The method according to claim 1, wherein the agent is 4-[[[[[[(2s)-4-[(3,4-dichlorophenyl)methyl]-2-morpholinyl]methyl]-amino]carbonyl]-amino]methyl]benzamide, or a pharmaceutically acceptable salt thereof.

11. The method according to claim 1, wherein the agent is N-[[(2S)-4-[(3,4-difluorophenyl)methyl]-2-morpholinyl]-methyl]-3-[(methylsulfonyl)amino]-benzeneacetamide, or a pharmaceutically acceptable salt thereof.

12. The method of claim 1, wherein the subject is mammalian.

13. The method of claim 1, wherein the subject is human.

14. The method of claim 1, further comprising administering to the subject additional therapeutic agents.

15. The method of claim 14, wherein the additional therapeutic agent is an anti-VEGF inhibitor selected from pazopanib, Lucentis®, Avastin®, and Aflibercept.

16. The method of claim 1, further comprising monitoring treatment by measuring visual acuity of said subject after administration of the pharmaceutical composition comprising an agent that inhibits CCR3.

17. (canceled)

18. (canceled)
